(19) United States
(12) Patent Application Publication
(10) Pub. No.: US 2004/0171941 A1
(43) Pub. Date: Sep. 2, 2004

(54) Blood flow amount estimating apparatus

(75) Inventors: Kiyoyuki Narimatsu, Komaki-shi (JP); Toshihiko Ogura, Komaki-shi (JP)

Correspondence Address: OLIFF & BERRIDGE, PLC, P.O. BOX 19928, ALEXANDRIA, VA, 22320, US

(73) Assignee: COLIN MEDICAL TECHNOLOGY CORPORATION, Komaki-shi (JP)

(21) Appl. No.: 10/780,734

(22) Filed: Feb. 19, 2004

(30) Foreign Application Priority Data
Feb. 28, 2003 (JP) 2003-053641

(51) Int. Cl. A61B 5/02

(52) U.S. Cl. 600/485; 600/493

(57) ABSTRACT

A blood flow amount estimating apparatus 10 which determines a cervical systolic blood pressure CBP(SYS) of a living subject, based on a brachial systolic blood pressure BBP(SYS) and a cervical pulse wave wc of the subject; determines, based on the cervical pulse wave and a heart sound of the subject, a pulse wave propagation velocity PWV as information related to sclerosis or hardness of a carotid artery 46 of the subject; and displays, in a two-dimensional graph 106 which is defined by a first axis 102 indicative of pulse wave propagation velocity PWV and a second axis 104 indicative of cervical systolic blood pressure CBP(SYS) and which indicates that blood flow amount changes with respective changes of cervical systolic blood pressure CBP(SYS) and pulse wave propagation velocity PVV, a symbol 108 representing the actually determined cervical systolic blood pressure CBP(SYS) and the actually determined pulse wave propagation velocity PWV, so that an observer can estimate, from the position of the symbol 108 in the graph 106, an amount of flow of blood in a cervical portion 38 of the subject.

### BACKGROUND OF THE INVENTION

1. Field of the Invention

The present invention relates to a blood flow amount estimating apparatus which non-invasively estimates an amount of flow of blood in a living subject.

2. Related Art Statement

Japanese Patent Publication No. 2000-201930 discloses, as an apparatus for non-invasively measuring an amount of flow of blood in a living subject, an ultrasound-using diagnosing apparatus. The ultrasound diagnosing apparatus includes a probe which is adapted to be pressed on a prescribed portion of a living subject and which has the functions of emitting an ultrasound beam toward the prescribed portion and receiving the ultrasound beam reflected from the portion (i.e., an ultrasound echo signal). The diagnosing apparatus determines, based on the ultrasound echo signal, a velocity of flow of blood in a blood vessel and a cross section area of the blood vessel, and additionally determines, based on the blood flow velocity and the blood vessel cross section area, an amount of flow of blood in the blood vessel.

It has been recognized that measurement of blood flow amount is clinically important. For example, an amount of flow of blood toward the brain of a person can be used to make a diagnosis on a disease of the brain. Thus, it has been proposed to measure a blood flow amount when a person undergoes a medical check. However, the ultrasound diagnosing apparatus is expensive. In addition, skill is needed to operate the apparatus, and a considerably long time is needed to perform measurement using the apparatus. Thus, it has not been clinically spread to make a diagnosis based on a blood flow amount measured using the apparatus.

### SUMMARY OF THE INVENTION

It is therefore an object of the present invention to provide a blood flow amount estimating apparatus which can easily estimate an amount of flow of blood in a living subject.

To this end, the Inventors have carried out extensive studies and found the following facts: If a blood vessel system is compared to an electric circuit, a blood flow corresponds to an electric current, and a blood pressure corresponds to an electric voltage. Although there would be various factors that correspond to a resistance to the blood flow, a degree of arteriosclerosis, i.e., a degree of hardness of an artery can be deemed as one of those factors, because arteriosclerosis is the inverse of blood-vessel compliance (i.e., degree of extensibility of blood vessel), that is, because, as blood-vessel compliance decreases, blood flow amount decreases. Thus, the Inventors have found that according to Ohm's law, a blood flow amount can be estimated based on a blood pressure and a degree of arteriosclerosis. The present invention has been developed on this finding.

According to the present invention, there is provided a blood flow amount estimating apparatus, comprising a blood pressure related information obtaining means for obtaining blood pressure related information that is related to a first blood pressure of a first portion of the subject; a pulse wave detecting device which detects a pulse wave from the first portion; an arteriosclerosis related information obtaining means for obtaining, based on the pulse wave detected by the pulse wave detecting device, arteriosclerosis related information that is related to arteriosclerosis or a degree of hardness of an artery of the first portion; an output device; and an output-device control means for controlling the output device to output a graph which has an axis indicative of blood pressure related information and an axis indicative of arteriosclerosis related information and indicates that blood flow amount changes with respective changes of blood pressure related information and arteriosclerosis related information, and additionally output, in the graph, a symbol representing the blood pressure related information obtained by the blood pressure related information obtaining means and the arteriosclerosis related information obtained by the arteriosclerosis related information obtaining means.

According to the present invention, the blood pressure related information obtaining means obtains the blood pressure related information related to the first blood pressure of the first portion of the subject; the arteriosclerosis related information obtaining means obtains, based on the pulse wave detected at the first portion, the arteriosclerosis related information related to arteriosclerosis, or degree of hardness of an artery, of the first portion; and the output-device control means controls the output device to output the graph which has the axis indicative of blood pressure related information and the axis indicative of arteriosclerosis related information and indicates that blood flow amount changes with respective changes of blood pressure related information and arteriosclerosis related information, and additionally output, in the graph, the symbol representing the blood pressure related information obtained by the blood pressure related information obtaining means and the arteriosclerosis related information obtained by the arteriosclerosis related information obtaining means. Therefore, an observer can estimate, from the position of the symbol in the graph, the amount of flow of blood in the first portion of the subject. Thus, based on the blood pressure related information, and the pulse wave, obtained with respect to, or from, the first portion of the subject, the blood flow amount at the first portion can be estimated. Since the blood pressure related information and the pulse wave can be easily measured or detected, the blood flow amount at the first portion can be easily estimated.

Here, preferably, the blood pressure related information obtaining means comprises a carotid pulse wave detecting device which is adapted to be pressed on a cervical portion of the subject as the first portion of the subject and detects a pressure pulse wave produced from a carotid artery of the cervical portion; a cuff which is adapted to be worn on a brachial portion of the subject as a second portion of the subject; a brachial blood pressure determining means for determining a plurality of brachial blood pressure values of the brachial portion, based on a first heartbeat synchronous signal detected from the brachial portion when a pressure in the cuff is changed; and a cervical blood pressure determining means for determining, as the blood pressure related information, a cervical blood pressure of the cervical portion, based on a minimum magnitude, an area-gravity-center magnitude, and a maximum magnitude of the carotid pulse wave detected by the carotid pulse wave detecting device, and the brachial blood pressure values determined by the brachial blood pressure determining means, and wherein the pulse wave detecting device comprises the carotid pulse wave detecting device of the blood pressure related information obtaining means. According to this feature, the cervical blood pressure determining means determines, as the blood pressure related information, the cervical blood pressure of the cervical portion as the first portion, and the arteriosclerosis related information obtaining means obtains the arteriosclerosis related information based on the carotid pulse wave detected from a carotid artery of the cervical portion as the first portion. Therefore, the observer can estimate, based on the position of the symbol in the graph outputted by the output device, an amount of flow of blood in the carotid artery toward the brain of the subject.

The arteriosclerosis related information may be a pulse wave propagation velocity, i.e., a velocity at which a pulse wave propagates between two portions of a living subject, or pulse wave propagation velocity related information that is related to the pulse wave propagation velocity; for example, a pulse wave propagation time that is needed by the pulse wave to propagate between the two portions of the subject. In the case where the pulse wave propagation velocity related information is obtained the arteriosclerosis related information, the present apparatus may further comprise a second heartbeat synchronous signal detecting device which detects a second heartbeat synchronous signal from a third portion of the subject, and the arteriosclerosis related information obtaining means may obtain, based on the pulse wave detected by the pulse wave detecting device and the second heartbeat synchronous signal detected by the second heartbeat synchronous signal detecting device, pulse wave propagation velocity related information that is related to a velocity at which the pulse wave propagates in the first portion of the subject.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a diagrammatic view showing a circuitry of a blood flow amount estimating apparatus to which the present invention is applied;

FIG. 2 is an illustrative view showing a state in which a pressure pulse wave detecting probe of the apparatus of FIG. 1 is worn on a cervical portion of a living subject;

FIG. 3 is an enlarged view of the pressure pulse wave detecting probe of FIG. 2, a portion of the probe being cut away;

FIG. 4 is a view for explaining a state in which an array of pressure sensing elements is provided in a pressing surface of a pressure pulse wave sensor shown in FIG. 3;

FIG. 5 is a diagrammatic view for explaining essential control functions of an electronic control device of the apparatus of FIG. 1;

FIG. 6 is a graph showing a pressure pulse wave represented by a pressure pulse wave signal SM2 (indicated at solid line) provided by a highest pressure detecting element, EM, and a pressure pulse wave represented by a pressure pulse wave signal SM2 (indicated at two-dot chain line) provided by a semiconductor pressure sensing element, E(x), positioned right above a non-flattened portion of the wall of a carotid artery;

FIG. 7 is a view showing a positional relationship between the highest pressure detecting element EM and the pressure sensing element E(x), and the carotid artery;

FIG. 8 is a graph showing a relationship between magnitude of carotid pulse wave, wc, and cervical blood pressure, CBP, that is determined by a cervical blood pressure determining means, shown in FIG. 5;

FIG. 9 is a view showing an example of a two-dimensional graph that is displayed on a display device under control of a display control means, shown in FIG. 5;

FIG. 10 is a flow chart for explaining more concretely a portion of the control functions of the control device, shown in FIG. 5; and

FIG. 11 is a flow chart for explaining more concretely another portion of the control functions of the control device, shown in FIG. 5.

### DETAILED DESCRIPTION OF PREFERRED EMBODIMENTS

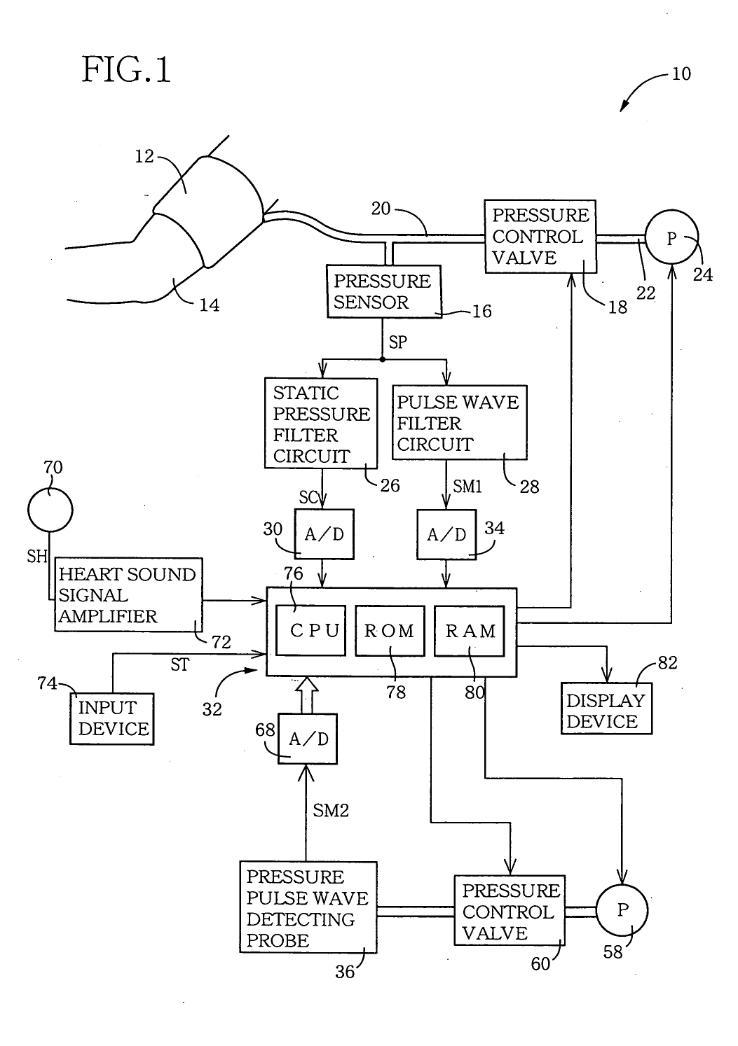

Hereinafter, there will be described a preferred embodiment of the present invention in detail by reference to the drawings. FIG. 1 is a diagrammatic view showing a circuitry of a blood flow amount estimating apparatus 10 to which the present invention is applied. The present apparatus 10 is used with a living subject who is taking a face-up position.

In FIG. 1, reference numeral 12 designates an inflatable cuff which includes a belt-like cloth bag and a rubber bag accommodated in the cloth bag and which is adapted to be wound around a brachial portion 14 of the subject. The cuff 12 is connected via a piping 20 to a pressure sensor 16 and a pressure control valve 18. The pressure control valve 18 is connected via a piping 22 to an air pump 24. The pressure control valve 18 adjusts a the pressure adjusted air to the cuff 12, or discharges the pressurized air from the cuff 12, so as to control an air pressure in the cuff 12.

The pressure sensor 16 detects the air pressure in the cuff 12, and supplies a pressure signal, SP, representing the detected air pressure, to a static pressure filter circuit 26 and a pulse wave filter circuit 28. The static pressure filter circuit 26 includes a low-pass filter which extracts, from the pressure signal SP, a cuff pressure signal, SC, representing a static component of the detected air pressure, i.e., a pressing pressure of the cuff 12 (hereinafter, referred to as the cuff pressure, PC). The filter circuit 26 supplies the cuff pressure signal SC to an electronic control device 32 via an A/D (analog-to-digital) converter 30. The pulse wave filter circuit 28 includes a band-pass filter which extracts, from the pressure signal SP, a cuff pulse wave signal, SM1, representing a cuff pulse wave as an oscillatory component of the detected air pressure. The filter circuit 28 supplies the cuff pulse wave signal SM1 to the control device 32 via an A/D converter 34. The cuff pulse wave represented by the cuff pulse wave signal SM1 is a pressure oscillation that is transmitted to the cuff 12 from a brachial artery, not shown, being pressed by the same, and will be referred to as a brachial pulse wave, where appropriate. The brachial pulse wave is a heartbeat synchronous signal which is produced in synchronism with the subject's heartbeat.

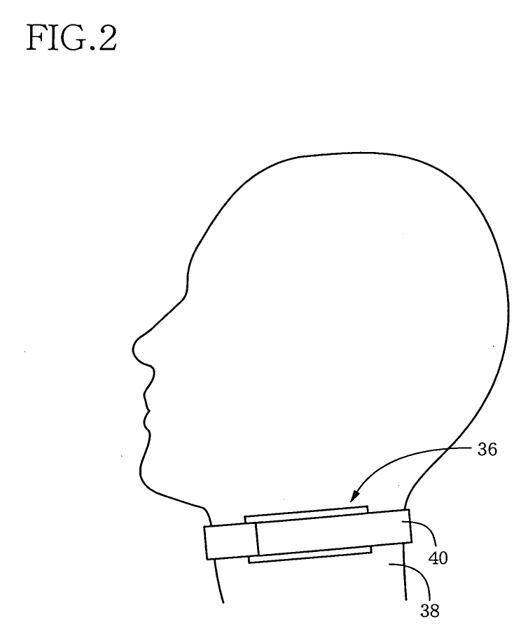

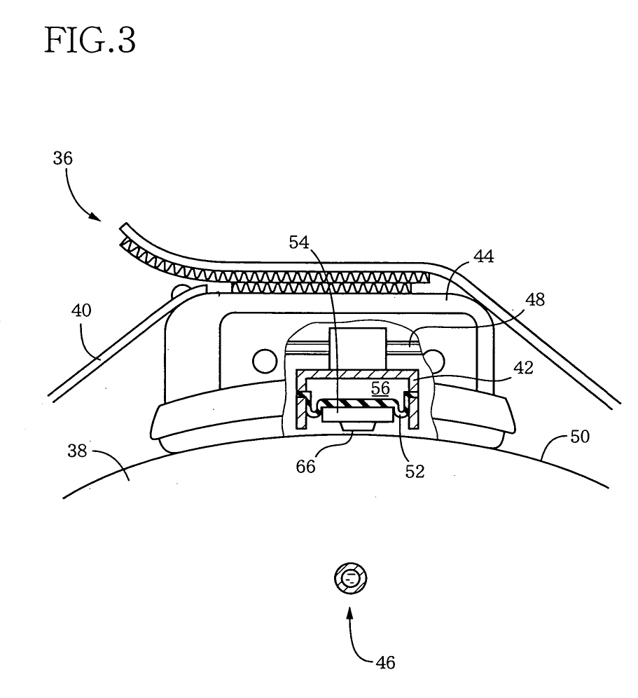

The blood flow amount estimating apparatus 10 includes a pressure pulse wave detecting probe 36, shown in FIG. 2, that functions as a carotid pulse wave detecting device. The pressure pulse wave detecting probe 36 is worn on a cervical portion 38 of the subject, as shown in FIG. 2, with the help of a band 40, so as to detect non-invasively a carotid pulse wave, wc, produced from a carotid artery 46. As shown in detail in FIG. 3, the pressure pulse wave detecting probe 36 includes a container-like sensor housing 42; a case 44 which accommodates the sensor housing 42; and a feed screw 48 which is threadedly engaged with the sensor housing 42 and is rotated by an electric motor, not shown, provided in the case 44 so as to move the sensor housing 42 in a widthwise direction of the carotid artery 46. With the help of the band 40, the pressure pulse wave detecting probe 36 is detachably attached to the cervical portion 38 of the subject, such that an open end of the sensor housing 42 is opposed to a body surface 50 of the cervical portion 38.

In addition, the detecting probe 36 includes a pressure pulse wave sensor 54 which is secured via a diaphragm 52 to an inner wall of the sensor housing 42, such that the sensor 54 is movable relative to the housing 42 and is advanceable out of the open end of the same 42. The sensor housing 42, the diaphragm 52, etc. cooperate with each other to define a pressure chamber 56, which is supplied with a pressurized air from an air pump 58 via a pressure control valve 60, as shown in FIG. 1, so that the pressure pulse wave sensor 54 is pressed against the body surface 50 with a pressing force corresponding to the air pressure in the pressure chamber 56.

The sensor housing 42 and the diaphragm 52 cooperate with each other to provide a pressing device 62 which presses the pressure pulse wave sensor 54 against the carotid artery 46, and the feed screw 48 and the not-shown electric motor cooperate with each other to provide a widthwise-direction moving device 64 which moves the pressure pulse wave sensor 54 in the widthwise direction of the carotid artery 46 and thereby changes a pressing position where the sensor 54 is pressed on the body surface 50.

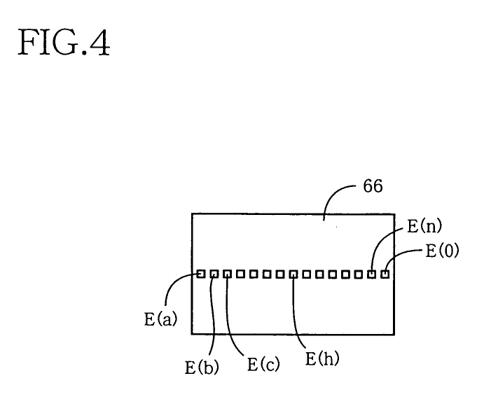

The pressure pulse wave sensor 54 has a pressing surface 66, and a number of semiconductor pressure-sensing elements (hereinafter, referred to as the “pressure sensing elements”) E which are arranged in the pressing surface 66 at a regular interval in the widthwise direction of the carotid artery 46, i.e., in the direction of movement of the sensor 54 parallel to the feed screw 48, over a length greater than the diameter of the carotid artery 46. For example, as shown in FIG. 4, fifteen pressure sensing elements E(a), E(b), . . . , E(o) are arranged at a regular interval of, e.g., 0.6 mm.

The pressure pulse wave detecting probe 36, constructed as described above, is pressed against the body surface 50 of the cervical portion 38 right above the carotid artery 46, so that the pressure pulse wave sensor 54 detects a pressure pulse wave (i.e., a carotid pulse wave wc) which is produced from the carotid artery 46 and is transmitted to the body surface 50, and supplies a pressure pulse wave signal SM2 representing the detected carotid pulse wave wc, to the control device 32 via an A/D converter 68, as shown in FIG. 1.

A heart sound microphone 70 is attached, with, e.g., an adhesive tape, not shown, to a chest of the subject. The heart sound microphone 56 functions as a heartbeat synchronous signal detecting device which detects heart sounds as a heartbeat synchronous signal. The microphone 70 incorporates a piezoelectric element, not shown, which converts the heart sounds produced from the heart of the subject, into an electric signal, i.e., a heart sound signal SH representing a waveform of the heart sounds. A heart sound signal amplifier 72 incorporates four sorts of filters, not shown, which cooperate with each other to attenuate a low-pitch component of the heart sounds that has a great energy, so as to allow clear recording of a high-pitch component of the heart sounds. The heart sound signal SH supplied from the heart sound microphone 56 is amplified and filtered by the heart sound signal amplifier 58, and then is supplied to the electronic control device 32 via an A/D converter, not shown.

An input device 74 includes a plurality of numeral keys, not shown, which are manually operable for inputting numerals representing a stature T of the subject, and supplies a stature signal ST representing the subject's stature T inputted through the keys, to the electronic control device 32.

The electronic control device 32 is provided by a so-called microcomputer including a CPU (central processing unit) 76, a ROM (read only memory) 78, a RAM (random access memory) 80, and an I/O (input and output) port, not shown. The CPU 76 processes signals according to the control programs pre-stored in the ROM 78 by utilizing the temporary-storage function of the RAM 80, and supplies drive signals via the I/O port to the air pumps 24, 58 and the pressure control valves 18, 60 so as to control the cuff pressure PC and the air pressure in the pressure chamber 56. Moreover, the CPU 76 determines, based on the signals supplied to the control device 32, a brachial blood pressure value, BBP, and a cervical blood pressure value, CBP, of the subject, and a pulse wave propagation velocity value, PWV, at which a pulse wave propagates between the subject's heart and the cervical portion 38. In addition, the CPU 76 operates a display device 82 as an output device to display a two-dimensional graph 106 defined by a first axis indicative of pulse wave propagation velocity PWV and a second axis indicative of cervical blood pressure CBP, and additionally display a determined-value symbol 108 representing the determined cervical blood pressure value CBP and the determined pulse wave propagation velocity value PWV, so that an observer such as a doctor or a nurse can estimate an amount of flow of blood based on a position of the symbol 108 in the graph 106 that represents the determined values CBP, PV.

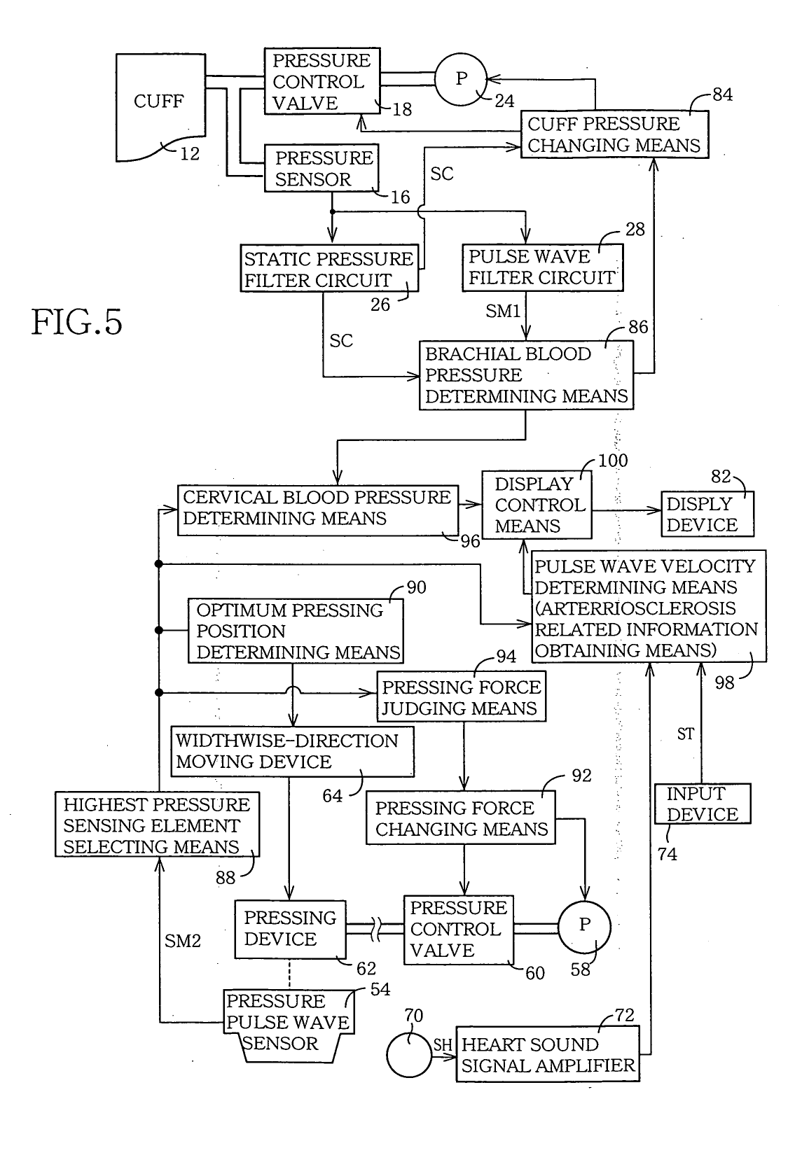

FIG. 5 is a diagrammatic view for explaining essential control functions of the electronic control device 32 of the blood flow amount estimating apparatus 10. A cuff pressure changing device or means 84 operates, according to a command signal supplied from a brachial blood pressure determining device or means 86, described later, and based on the cuff pressure signal SC supplied from the static pressure filter circuit 26, the pressure control valve 18 and the air pump 24 so as to change the cuff pressure PC as follows: First, the cuff pressure changing means 84 quickly increases the cuff pressure PC up to a prescribed target pressure PCM (e.g., 180 mmHg) that would be higher than a systolic blood pressure BBP(SYS) of the brachial portion 14 of the subject and, subsequently, slowly decreases the cuff pressure PC at a low rate of, e.g., 3 mmHg/sec. After a brachial diastolic blood pressure BBP(DIA) of the subject is determined by the brachial blood pressure determining means 86, the changing means 84 releases the cuff pressure PC down to atmospheric pressure.

The brachial blood pressure determining device or means 86 determines, based on the cuff pressure signal SC continuously supplied from the static pressure filter circuit 26, and the cuff pulse wave signal SM1 continuously supplied from the pulse wave filter circuit 28, each during the slow decreasing of the cuff pressure PC under the control of the cuff pressure changing means 84, a systolic blood pressure BBP(SYS), a mean blood pressure BBP(MEAN), and a diastolic blood pressure BBP(DIA) of the brachial portion 14 of the subject, according to a well-known oscillometric algorithm.

A highest pressure detecting element selecting device or means 88 determines, as a highest pressure detecting element EM, one of the pressure sensing elements E of the pressure pulse wave sensor 54 that detects the highest pressure of the respective pressure values detected by all the elements E. More specifically described, the selecting means 88 determines the greatest one of respective magnitudes of respective peak points of the respective pressure pulse waves detected by all the elements E, and determines, as the highest pressure detecting element EM, one of the pressure sensing elements E that provides the greatest magnitude. The highest pressure detecting element EM is one of the elements E that is positioned right above the carotid artery 46.

An optimum pressing position determining device or means 90 judges whether a prescribed pressing position changing condition is satisfied, i.e., whether the highest pressure detecting element EM of the sensor 54 is positioned in one of prescribed opposite end portions of the array of pressure sensing elements E. Each of the prescribed opposite end portions of the array of elements E may be a range having a prescribed length including a corresponding one of the opposite ends of the array of elements E, or a range accommodating a prescribed number of elements E including a corresponding one of the respective elements E located at the opposite ends of the array. When this pressing position changing condition is satisfied, e.g., when the sensor 54 is initially worn on the subject, the optimum pressing position determining means 90 carries out the following pressing position changing operation: After the pressing device 62 once moves the pressure pulse wave sensor 54 away from the body surface 50, the widthwise direction moving device 64 moves the pressing device 62 and the sensor 54 over a prescribed distance, and then the pressing device 62 again presses the sensor 54 with a prescribed, considerably small first pressing force, HDP1. In this state, the determining means 90 judges again whether the prescribed pressing position changing condition is satisfied. The determining means 90 repeats carrying out the above described operation and judgment till the pressing position changing condition is not satisfied any longer, preferably till the highest pressure detecting element EM is positioned in a prescribed middle portion of the array of elements E. The length, or number of elements E, employed for defining each of the opposite end portions of the array of elements E is prescribed based on the diameter of an artery (i.e., the carotid artery 46) to be pressed by the pressure pulse wave sensor 54, and is, e.g., one fourth of the diameter.

A pressing force changing device or means 92 changes, after the optimum pressing position determining means 90 positions the pressure pulse wave sensor 54 at the optimum pressing position, a pressing force HDP (i.e., a hold-down pressure) applied by the pressing device 62 to the sensor 54, within a prescribed pressing force range, either stepwise in response to each heartbeat of the subject or continuously at a prescribed, response to each heartbeat of the subject or continuously at a prescribed, considerably low rate. While the pressing force HDP is changed, if a pressing force judging device or means 94, described below, judges that a current pressing force HDP applied to the sensor 54 is appropriate, then the changing means 92 determines the current HDP as an optimum pressing force HDPO, and maintains the pressing force of the pressing device 62, at the thus determined optimum pressing force HDPO.

The pressing force judging means 94 judges whether the current pressing force HDP applied to the pressure pulse wave sensor 54 is appropriate, based on the pressure pulse wave detected by the highest pressure detecting element EM selected by the highest pressure detecting element selecting means 88, and the respective pressure pulse waves detected by two pressure sensing elements E (hereinafter, referred as to the “comparison elements EC”) that are distant from the element EM by a prescribed distance in respective directions from the element EM toward the opposite ends of the array of elements E. More specifically described, the judging means 94 determines a time difference, ΔT, between a time of detection of a prescribed periodic point of a heartbeat synchronous pulse of the pressure pulse wave detected by the highest pressure detecting element EM and a time of detection of a prescribed periodic point of a corresponding heartbeat synchronous pulse of the pressure pulse wave detected by at least one of the two comparison elements EC, and judges whether the pressing force HDP is appropriate based on the thus determined time difference ΔT. The respective prescribed periodic points of respective heartbeat synchronous pulses of the pressure pulse waves may be respective rising points, respective peak points, or respective dicroitic notches of the respective pulses.

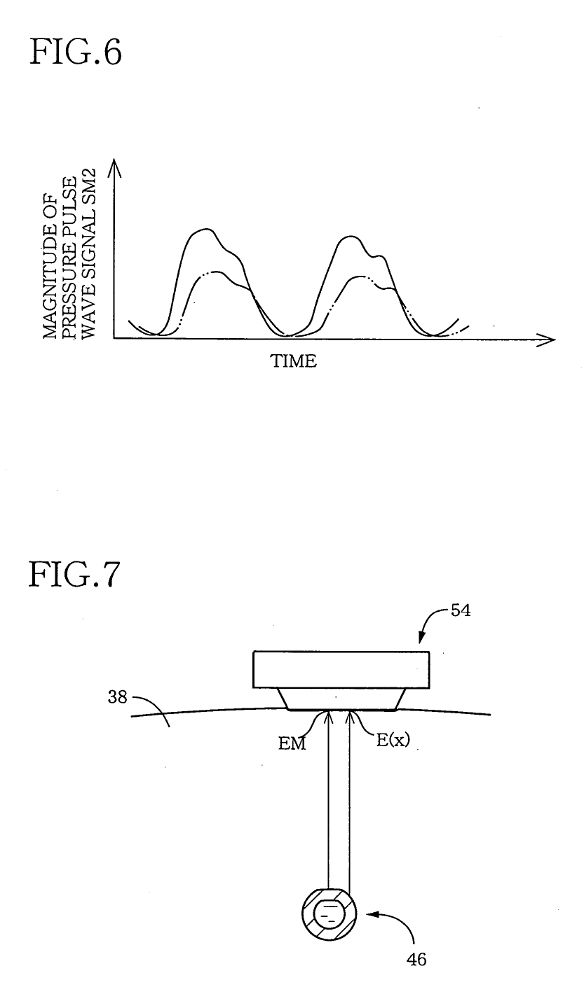

The reason why the above indicated time difference ΔT can be used to judge whether the pressing force HDP applied to the pressure pulse wave sensor 54 is appropriate, is as follows: FIG. 6 shows a pressure pulse wave represented by the pressure pulse wave signal SM2 (indicated at solid line) provided by the highest pressure detecting element EM positioned right above a flattened portion of the wall of the carotid artery 46 and a pressure pulse wave represented by the pressure pulse wave signal SM2 (indicated at two-dot-chain line) provided by a pressure sensing element, E(x), positioned right above a non-flattened portion of the arterial wall; and detecting element EM and the pressure sensing element E(x), and the carotid artery 46. As shown in FIG. 6, a phase of a heartbeat synchronous pulse of the pressure pulse wave detected by the pressure sensing element E(x) is delayed from a phase of a corresponding heartbeat synchronous pulse of the pressure pulse wave detected by the highest pressure detecting element EM, because the pressure pulse wave detected by the pressure sensing element E(x) is influenced by viscoelasticity of the non-flattened portion of the arterial wall but the pressure pulse wave detected by the highest pressure detecting element EM is not influenced by viscoelasticity of the flattened portion of the arterial wall. Therefore, if the phase of the pressure pulse wave detected by the pressure sensing element E(x) is not delayed, or is delayed by only a short time, if any, from the phase of the pressure pulse wave detected by the highest pressure detecting element EM, then it can be said that the pressure sensing element E(x) is positioned right above a substantially flattened portion of the wall of the carotid artery 46 (or it can be said that a portion of the wall of the carotid artery 46 is substantially flattened by the pressing force applied to the sensor 54).

Therefore, if the time difference ΔT between the time of detection of the prescribed periodic point of the pressure pulse wave detected by the highest pressure detecting element EM and the time of detection of the prescribed periodic point of the pressure pulse wave detected by at least one of the two comparison elements EC, is smaller than an upper-limit time, TH1, that is experimentally determined in advance, then it can be judged that the carotid artery 46 is in the state in which a portion of the wall of the artery 46 is substantially flattened by the pressing force HDP applied to the pressure pulse wave sensor 54, that is, that the current pressing force HDP applied to the sensor 54 is appropriate. The distance between the highest pressure detecting element EM and each of the two comparison elements EC is so prescribed as to be shorter than the diameter of the artery 46 (e.g., so as to be equal to one fifth of the diameter of the artery 46).

A cervical blood pressure determining device or means 96 first determines, based on the pressure pulse wave signal SM2 supplied from the highest pressure detecting element EM in the state in which it has already been judged by the pressing force judging means 94 that the current pressing force HDP applied to the sensor 54 is appropriate, a minimum magnitude, a, an area gravity center magnitude, b, and a maximum magnitude, c, of a unit length of the carotid pulse wave wc represented by the signal SM2. The unit length is defined in terms of heartbeat, such as one heartbeat or several heartbeats, or in terms of time, such as several seconds or several tens of seconds. The area gravity center magnitude b of the unit length is defined as an average magnitude of one-heartbeat length (i.e., a heartbeat synchronous pulse) of the cervical pulse wave wc; for example, the magnitude b may be calculated by integrating the magnitude of heartbeat synchronous pulse of the cervical pulse wave wc and dividing the thus obtained integral value by a time period of the heartbeat synchronous pulse, i.e., a pulse period T.

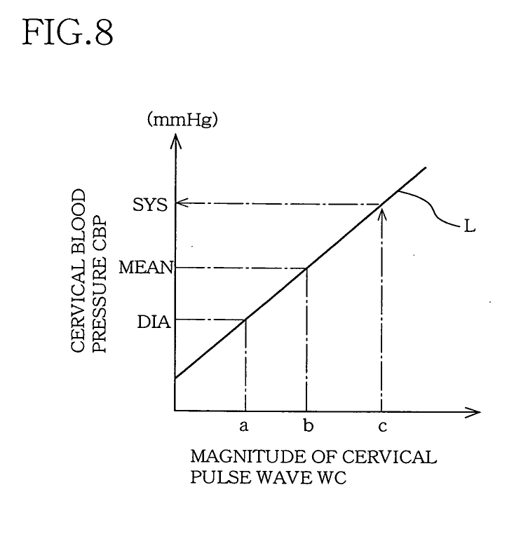

Meanwhile, it is known that when a living person rests in a face-up position, a brachial mean blood pressure BBP(MEAN) and a brachial diastolic blood pressure BBP(DIA) of the person are substantially equal to a cervical mean blood pressure CBP(MEAN) and a cervical diastolic blood pressure CBP(DIA) of the same person, respectively. According to this fact, the cervical blood pressure determining means 96 determines, based on the brachial mean and diastolic blood pressure values BBP(MEAN), BBP(DIA) and the area gravity center magnitude b and minimum magnitude a of the cervical pulse wave wc, a straight line, L, shown in FIG. 8, that represents a relationship between magnitude of cervical pulse wave wc and cervical blood pressure CBP.

In addition, the cervical blood pressure determining means 96 determines or estimates, based on the straight line L and the maximum magnitude c of the cervical pulse wave wc, a cervical systolic blood pressure CBP(SYS) of the subject. In the present embodiment, the thus determined or estimated cervical systolic blood pressure CBP(SYS) provides a set of blood pressure related information; and the cervical blood pressure determining means 96 cooperates with the cuff 12, the brachial blood pressure determining means 86, and the pressure pulse wave detecting probe 36 to provide a blood pressure related information obtaining means.

A pulse wave velocity determining device or means 98 determines, based on the heart sound signal SH continuously supplied from the heart sound microphone 70 and the pressure pulse wave signal SM2 continuously supplied from the highest pressure detecting element EM, each in the state in which the pressing force HDP applied to the pressure pulse wave sensor 54 is maintained at the optimum pressing force HDPO by the pressing force changing means 92, a time difference between a time of detection of a prescribed periodic point on the heart sound waveform represented by the heart sound signal SH and a time of detection of a prescribed periodic point on the cervical pulse wave wc represented by the pressure pulse wave signal SM2 which periodic point corresponds to the periodic point of the heart sound waveform. This time difference means a pulse wave propagation time DT that is needed for the pulse wave wc to propagate from the subject's heart to the cervical portion 38.

In addition, the pulse wave velocity determining means 98 replaces the following expression (1) defining a pre-stored relationship between stature T and propagation distance d, with the subject's stature T supplied from the input device 74, and thereby determines a propagation distance d between the patient's heart and the cervical portion 38, and subsequently replaces the following expression (2) with the thus determined propagation distance d and the above described pulse wave propagation time DT, and thereby determines a pulse wave propagation velocity PVVV (cm/sec):

d=αT+β Expression (1)

where α, β are experimentally obtained constants,

PWV=d/DT Expression (2)

In the present embodiment, the thus determined pulse wave propagation velocity PWV provides a set of arteriosclerosis related information; and the pulse wave velocity determining means 98 provides an arteriosclerosis related information obtaining means.

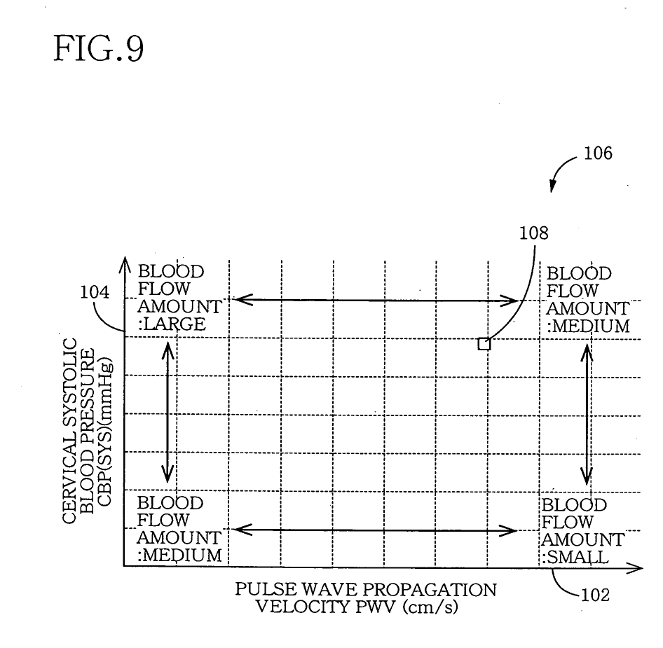

A display control device or means 100 operates the display device 82 to display a two-dimensional graph 106, shown in FIG. 9, that is defined by a first axis indicative of pulse wave propagation velocity PWV and a second axis indicative of cervical systolic blood pressure CBP(SYS), and additionally display, in the graph 106, a determined-value symbol 108 representing the pulse wave propagation velocity PWV determined by the pulse wave velocity determining means 96 and the cervical systolic blood pressure CBP(SYS) determined by the cervical blood pressure determining means 94.

In addition, the display control means 100 operates the display device 82 to display, in the two dimensional graph 106, indications that as pulse wave propagation velocity PUV increases, blood flow amount decreases and that as cervical systolic blood pressure CBP(SYS) increases, blood flow amount increases. Those indications, displayed in the graph 106, that as pulse wave propagation velocity PWV increases, blood flow amount decreases and, as cervical systolic blood pressure CBP(SYS) increases, blood flow amount increases can be proved by comparing a vascular system of a living person to an electric circuit, as follows: Since pulse wave propagation velocity PWV as an index of arteriosclerosis is compared to impedance, it can be said that as pulse wave propagation velocity PUV increases, impedance increases and, since blood pressure BP is compared to electric voltage, it can be said according to Ohm's law that blood flow amount corresponding to electric current is in proportion to the blood pressure BP and is in inverse proportion to the pulse wave propagation velocity PWV.

Since the display device 82 displays, in the two dimensional graph 106 indicating the relationship between pulse wave propagation velocity PWV and blood flow amount and the relationship between cervical systolic blood pressure CBP(SYS) and blood flow amount, the determined-value symbol 108 representing the pulse wave propagation velocity PWV actually determined by the pulse wave velocity determining means 96 and the cervical systolic blood pressure CBP(SYS) actually determined by the cervical blood pressure determining means 94, an observer such as a doctor or a nurse can estimate, from the position of the symbol 108 in the graph 106, an amount of flow of blood in the cervical portion 38 toward the subject's brain.

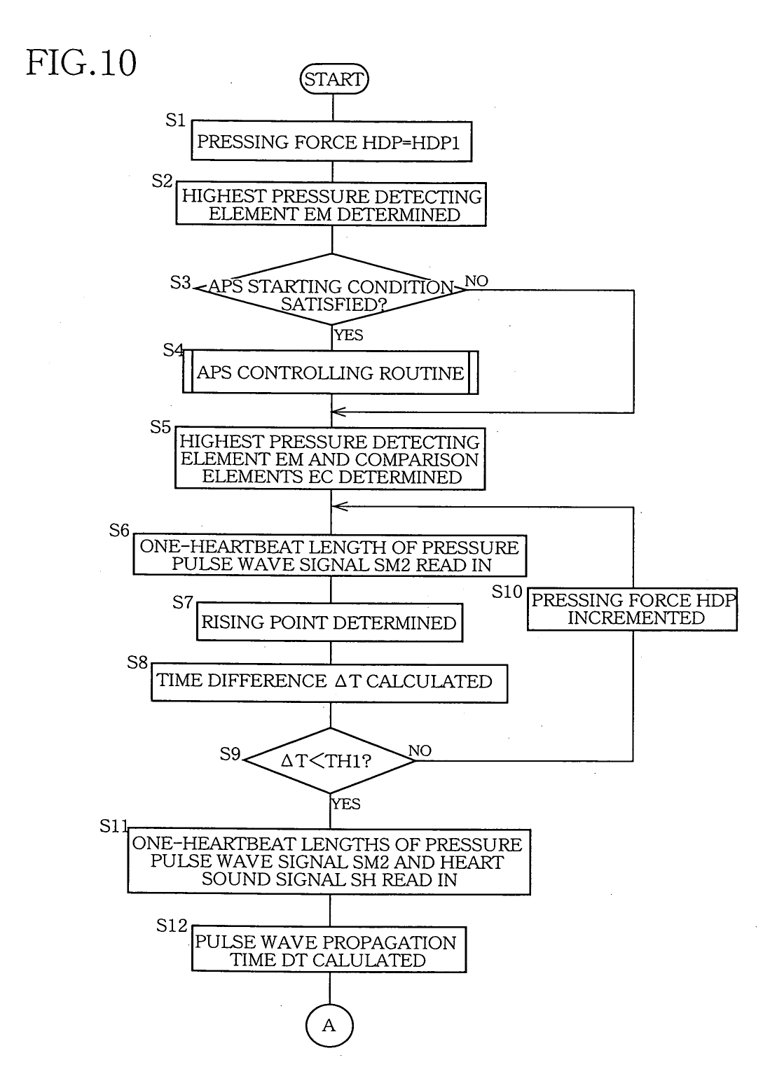

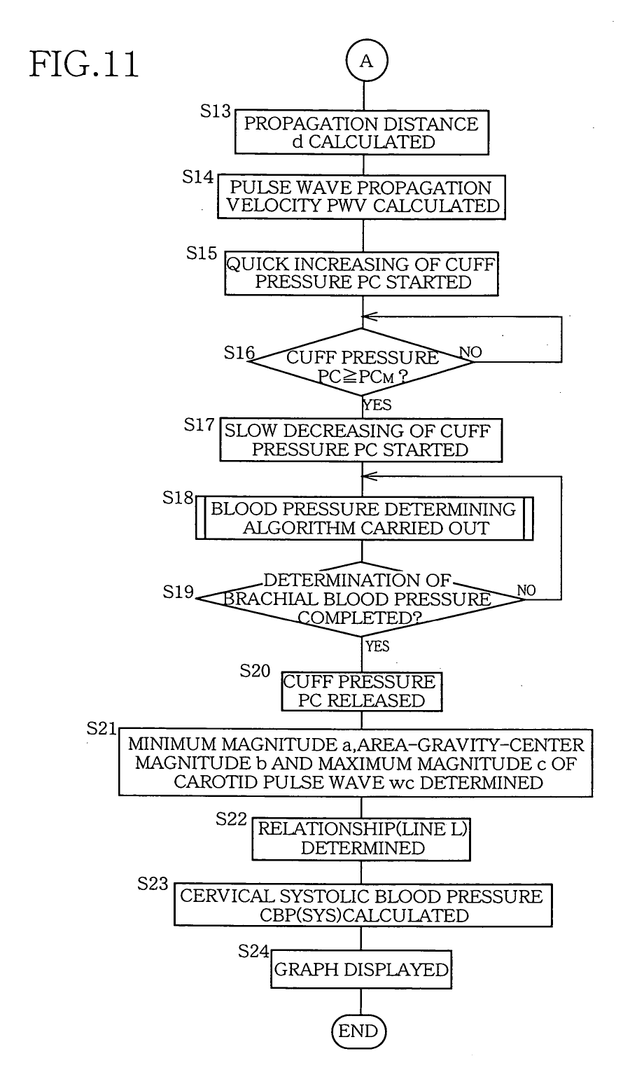

FIGS. 10 and 11 are flow charts representing the control functions of the electronic control device 32, shown in the diagrammatic view of FIG. 5. Those flow charts are started upon operation of a start button, not shown, on the assumption that the stature signal ST representing the subject's stature T has been supplied from the input device 74 to the control device 32.

In FIG. 10, first, the control device 32 carries out Step S1 corresponding to the pressing-force changing means 92. At Step S1, the control device operates the pressing device 62 to change the pressure in the pressure chamber 56 so that the pressing force HDP applied to the pressure pulse wave sensor 54 is changed to the prescribed first pressing force HDP1. The first pressing force HDP1 is so prescribed as to be sufficiently smaller than an average optimum pressing force HDPO, and is experimentally determined, in advance, to assure that the control device can accurately determine respective magnitudes of respective peak points of the respective pressure pulse waves detected by the respective pressure sensing elements E, based on the respective pressure pulse wave signals SM2 supplied from the elements E.

Subsequently, the control of the control device goes to Step S2 corresponding to the highest pressure detecting element determining means 88. At Step S2, the control device reads in respective one-heartbeat lengths of the respective pressure pulse wave signals SM2 supplied from the pressure sensing elements E, determines respective peak points of the respective pressure pulse waves represented by the respective one-heartbeat lengths of the pressure pulse wave signals SM2, determines respective magnitudes of the thus determined peak points, and determines one of the pressure sensing elements E that provides the greatest peak magnitude, as a highest pressure detecting element EM.

Then, the control goes to Steps S3 and S4 corresponding to the optimum pressing position determining means 90. First, at Step S3, the control device judges whether a prescribed pressing-position changing condition (i.e., an APS starting condition) is satisfied, i.e., whether the highest pressure detecting element EM is positioned in one of the prescribed opposite end portions of the array of pressure sensing elements E. If a negative judgment is made at Step S3, the control jumps to Step S5 and the following steps, described later.

On the other hand, if a positive judgment is made at Step S3, that is, if the position of the pressure pulse wave sensor 54 relative to the carotid artery 46 is not appropriate, the control goes to Step S4 to carry out an APS controlling routine. More specifically described, the highest pressure detecting element EM is moved to an optimum pressing position where the highest pressure detecting element EM is located at substantially the middle of the array of pressure sensing elements E, in such a manner that after the pressing device 62 once moves the pressure pulse wave sensor 54 away from the body surface 50, the widthwise direction moving device 64 moves the pressing device 62 and the sensor 54 over a prescribed distance, and then the pressing device 62 again presses the sensor 54 with the above-described first pressing force HDP1. In this state, the control device judges again whether the highest pressure detecting element EM is positioned in a prescribed middle portion of the array of elements E. The control device repeats carrying out those operation and judgment till a positive judgment is made at Step S4.

After the pressure pulse wave sensor 54 is positioned at the optimum pressing position, the control goes to Step S5 corresponding to the highest pressure detecting element determining means 88. At Step S5, the control device determines a highest pressure detecting element EM in the same manner as employed at Step S2, and additionally determines two pressure sensing elements E on either side of, and next to, the highest pressure detecting element EM, as comparison elements EC.

Subsequently, the control goes to Steps S6 through S9 corresponding to the pressing force judging means 94. First, at Step S6, the control device reads in, at a prescribed sampling period, Ts, the respective pressure pulse wave signals SM2 supplied from the there pressure sensing elements EM, EC, and obtains respective one-heartbeat lengths of the respective signals SM2. Subsequently, at Step S7, the control device determines, as a rising point, a point where a rate of increase of amplitude of the pressure pulse wave represented by the pressure pulse wave signal SM2 supplied from the highest pressure detecting element EM, read in at Step S6, takes a maximum value and additionally determines, as a reference time, Tst, a time of detection of the rising point. In the same manner, the control device determines respective rising points with respect to the respective pressure pulse wave signals SM2 supplied from the two comparison elements EC, and determines, as respective comparison times Tco, respective times of detection of those rising points.

Subsequently, at Step S8, the control device calculates a time difference, ΔT, between the reference time Tst determined at Step S7 and each of the two comparison times Tco determined at the same step. Thus, the two time difference values ΔT are obtained each as an absolute value. Subsequently, at Step S9, the control device judges whether each of the two time difference values ΔT calculated at Step S8 is smaller than an upper-limit time, TH1, that is so prescribed as to be from one fold to three folds longer than the sampling period Ts. Since the two comparison elements EC are selected and the two time difference values ΔT are calculated, it is preferred, at Step S9, to judge whether each of the two time difference values ΔT is smaller than the upper limit time TH1. However, Step S9 may be modified such that the control device judges whether at least one of the two time difference values AT is smaller than the upper limit time TH1.

If a negative judgment is made at Step S9, the control goes to Step S10 corresponding to the pressing force changing means 90. At Step S10, the control device operates the pressing device 62 to press the pressure pulse wave sensor 54 with the pressing force HDP increased by a prescribed amount, and then the control goes back to Step S6 and the following steps. Meanwhile, if a positive judgment is made at Step S9, that is, if the pressing force HDP applied to the sensor 54 is appropriate, the control goes to Step S11. At Step S11, the control device reads in, at the sampling period Ts, the pressure pulse wave signal SM2 supplied from the highest pressure detecting element EM and the heart sound signal SH supplied from the heart sound microphone 70 via the heart sound signal amplifier 72, and obtains respective one-heartbeat lengths of the signals SM2, SH.

Subsequently, the control goes to Steps S12, S13, and S14 corresponding to the pulse wave velocity determining means 98. First, at Step S12, the control device determines a starting point of a heart sound II on the heart sound waveform represented by the heart sound signal read in at Step S11, and additionally determines a dichroitic notch on the carotid pulse wave wc represented by the pressure pulse wave signal SM2 read in at the same step. The starting point of heart sound II and the dichroitic notch are respective periodic points of the heart sound waveform and the carotid pulse wave that correspond to each other. In addition, the control device determines, as a pulse wave propagation time DT, a time difference between the starting point of heart sound II and the dichroitic notch. Subsequently, at Step S13, the control device replaces the above-indicated Expression 1 with the subject's stature T represented by the stature signal ST that has already been supplied to the control device, and thereby calculates a propagation distance d. Then, at Step S14, the control device replaces the above-indicated Expression 2 with the pulse wave propagation time DT determined at Step S12 and the propagation distance d calculated at Step S13, and thereby calculates a pulse wave propagation velocity PWV.

Subsequently, at Step S15, the control device operates the pressure control valve 18 so as to start quickly increasing the cuff pressure PC. Subsequently, at Step S16, the control device judges whether the cuff pressure PC has exceeded a prescribed target pressure PCM equal to 180 mmHg. Step S16 is repeated until a positive judgment is made, while the cuff pressure PC is quickly increased. Meanwhile, if a positive judgment is made at Step S16, the control goes to Step S17 to stop the air pump 24 and operate the pressure control valve 18 so as to start slowly decreasing the cuff pressure PC at a rate of about 3 mmHg/sec.

Then, the control goes to Steps S18 and S19 corresponding to the brachial blood pressure determining means 86. At Step S18, the control device determines, based on change of respective amplitudes of successive heartbeat-synchronous pulses of the brachial pulse wave represented by the cuff pulse wave signal SM1 continuously obtained during the slow decreasing of the cuff pressure PC, a brachial systolic blood pressure BBP(SYS), a brachial mean blood pressure BBP(MEAN), and a brachial diastolic blood pressure BBP(DIA) of the brachial portion 14 of the subject, according to well-known oscillometric blood pressure determining algorithm. Then, at Step S19, the control device judges whether the determination of the brachial blood pressure values BBP has been completed at Step S18.

Step S18 is repeated until a positive judgment is made at Step S19, while the blood pressure determining algorithm is continued. Meanwhile, if a positive judgment is made at Step S19, the control goes to Step S20 to operate the pressure control valve 18 to decrease the cuff pressure PC to an atmospheric pressure. In the present flow charts Steps S15, S16, S17, and S22 correspond to the cuff pressure changing means 84.

Then, the control goes to Steps S21, S22, and S23 corresponding to the cervical blood pressure determining means 96. First, at Step S21, the control device determines a minimum magnitude a and a maximum magnitude c of a heartbeat-synchronous pulse of the carotid pulse wave wc that is represented by the one-heartbeat length of the pressure pulse wave signal SM2, read in at Step S11 of FIG. 10, and additionally determines, as an area-gravity-center magnitude b of the carotid pulse wave wc, an average magnitude of the heartbeat-synchronous pulse of the carotid pulse wave wc.

Subsequently, at Step S22, the control device determines, based on the minimum magnitude a and the area gravity center magnitude b of the carotid pulse wave wc, determined at Step S21, and the brachial diastolic and mean blood pressure values CBP(DIA), CBP(MEAN) determined at Steps S18 and S19, a relationship between magnitude of carotid pulse wave wc and cervical blood pressure CBP, as shown in the graph of FIG. 8. Subsequently, at Step S23, the control device determines or estimates a cervical systolic blood pressure value CBP(SYS) of the cervical portion 38 of the subject, based on the maximum magnitude c of the carotid pulse wave wc, determined at Step S21, according to the relationship determined at Step S22.

Then, the control goes to Step S24 corresponding to the display control means 100. At Step S24, the control device controls the display device 82 to display, in a two-dimensional graph 106 which is defined by a first axis 102 indicative of pulse wave propagation velocity PWV and a second axis 104 indicative of cervical systolic blood pressure CBP(SYS) and which indicates that blood flow amount changes with respective changes of pulse wave propagation velocity PWV and cervical systolic blood pressure CBP(SYS), a symbol 108 representing the pulse wave propagation velocity PWV calculated at Step S14 and the cervical systolic blood pressure CBP(SYS) calculated at Step S23, so that an observer can estimate, from the position of the symbol 108 in the graph 106, a blood flow amount in the cervical portion 38 of the subject.

In the above-described embodiment, the cervical blood pressure determining means 96 (Steps S21, S22, S23) determines, based on the brachial blood pressure values BBP of the brachial portion 14 and the carotid pulse wave wc detected from the cervical portion 38, the cervical systolic blood pressure value CBP(SYS); the pulse wave velocity determining means 98 (Steps S12, S13, S14) determines, based on the carotid pulse wave wc and the heart sound, the pulse wave propagation velocity PWV as the information related to sclerosis or hardening of the carotid artery 46; and the display control means 100 (Step S24) controls the display device 82 to display the two dimensional graph 106 which has the first axis 102 indicative of cervical systolic blood pressure CBP(SYS) and the second axis 104 indicative of pulse wave propagation velocity PWV and indicates that blood flow amount changes with respective changes of cervical systolic blood pressure CBP(SYS) and pulse wave propagation velocity PWV;, and additionally display, in the two dimensional graph 106, the symbol 108 representing the cervical systolic blood pressure value CBP(SYS) determined by the cervical blood pressure determining means 96 (Steps S21 through S23) and the pulse wave propagation velocity PVUV determined by the pulse wave velocity determining means 98 (Steps S12 through S14). Therefore, the observer can estimate, from the position of the symbol 108 in the two dimensional graph 106, the amount of flow of blood in the cervical portion 38 or the carotid artery 46 toward the subject's brain. Thus, based on the brachial blood pressure values BBP measured at the brachial portion 14 of the subject, the carotid pulse wave wc detected at the cervical portion 38, and the heart sound detected at the heart, the blood flow amount at the cervical portion 38 can be estimated. Since the brachial blood pressure values BBP, the carotid pulse wave wc, and the heart sound can be easily measured or detected, the blood flow amount at the cervical portion 38 can be easily estimated.

While the present invention has been described in its preferred embodiment by reference to the drawings, it is to be understood that the invention may otherwise be embodied.

For example, in the above-described embodiment, the blood flow amount at the cervical portion 38 is estimated. However, a blood flow amount can be estimated at a different portion of the subject, such as the brachial portion 14, an ankle, or a femoral portion.

Also, in the above-described embodiment, the systolic blood pressure value BP(SYS) is obtained as the blood pressure related information. However, a diastolic blood pressure BP(DIA), a mean blood pressure BP(MEAN), or a pulse pressure (i.e., a difference between a systolic blood pressure BP(SYS) and a diastolic blood pressure BP(DIA)) may be obtained as the blood pressure related information. In addition, the blood pressure related information may be obtained in such a manner that a first blood pressure is measured at a first portion (e.g., the cervical portion 38) of the subject, a second blood pressure is measured at a second portion (e.g., the brachial portion 14) of the subject, and a difference between the first blood pressure and the second blood pressure BP(DIA)) is obtained as the information

Also, in the above-described embodiment, the pulse wave

Also, in the above-described embodiment, the pulse wave propagation velocity PWV is obtained as the arteriosclerosis related information. However, an augmentation index AI is known as a sort of arteriosclerosis related information. The augmentation index AI indicates a proportion of a reflected-wave component of a pulse wave to an incident-wave component of the same, and is calculated by dividing, with a pulse pressure PP of the pulse wave, a pressure difference, ΔP, obtained by subtracting a pressure corresponding to a magnitude of the pulse wave at the time of detection of a peak point of the incident-wave component from a pressure corresponding to a magnitude of the pulse wave at the time of detection of a peak point of the reflected-wave component. Thus, the augmentation index AI may be obtained as the arteriosclerosis related information. When the augmentation index AI is determined, an inflection point or a local maximum point between a rising point and a peak point of the pulse wave may be determined as the peak point of the incident-wave component; and the first local maximum point of the pulse wave following the peak point of the incident-wave component may be determined as the peak point of the reflected-wave component.

While the present invention has been described in detail in its preferred embodiments by reference to the drawings, it is to be understood that the present invention may be embodied with other changes and improvements that may occur to a person skilled in the art without departing from the spirit and scope of the invention defined in the appended claims.

What is claimed is:

1. A blood flow amount estimating apparatus, comprising:
a blood pressure related information obtaining means for obtaining blood pressure related information that is related to a first blood pressure of a first portion of the subject;
a pulse wave detecting device which detects a pulse wave from the first portion;
an arteriosclerosis related information obtaining means for obtaining, based on the pulse wave detected by the pulse wave detecting device, arteriosclerosis related information that is related to arteriosclerosis;
an output device; and
an output-device control means for controlling the output device to output a graph which has an axis indicative of blood pressure related information and an axis indicative of arteriosclerosis related information and indicates that blood flow amount changes with respective changes of blood pressure related information and arteriosclerosis related information, and additionally output, in the graph, a symbol representing the blood pressure related information obtained by the blood pressure related information obtaining means and the arteriosclerosis related information obtained by the arteriosclerosis related information obtaining means.

2. The apparatus according to claim 1, wherein the blood pressure related information obtaining means comprises:
a carotid pulse wave detecting device which is adapted to be pressed on a cervical portion of the subject as the first portion of the subject and detects a pressure pulse wave produced from a carotid artery of the cervical portion;
a cuff which is adapted to be worn on a brachial portion of the subject as a second portion of the subject;
a brachial blood pressure determining means for determining a plurality of brachial blood pressure values of the brachial portion, based on a first heartbeat synchronous signal detected from the brachial portion when a pressure in the cuff is changed; and
a cervical blood pressure determining means for determining, as the blood pressure related information, a cervical blood pressure of the cervical portion, based on a minimum magnitude, an area-gravity-center magnitude, and a maximum magnitude of the carotid pulse wave detected by the carotid pulse wave detecting device, and the brachial blood pressure values determined by the brachial blood pressure determining means, and
wherein the pulse wave detecting device comprises the carotid pulse wave detecting device of the blood pressure related information obtaining means.

3. The apparatus according to claim 1, further comprising a second heartbeat synchronous signal detecting device which detects a second heartbeat synchronous signal from a third portion of the subject, wherein the arteriosclerosis related information obtaining means obtains, based on the pulse wave detected by the pulse wave detecting device and the second heartbeat synchronous signal detected by the second heartbeat synchronous signal detecting device, the arteriosclerosis related information comprising pulse wave propagation velocity related information that is related to a velocity at which the pulse wave propagates in the first portion of the subject.

4. The apparatus according to claim 1, wherein the blood pressure related information obtaining means comprises a blood pressure measuring device which non-invasively measures a second blood pressure of a second portion of the subject, wherein the blood pressure related information obtaining means obtaining, based on the second blood pressure measured by the blood pressure measuring device, the blood pressure related information that is related to the first blood pressure of the first portion of the subject.

5. The apparatus according to claim 1, wherein the output device comprises a display device which displays the graph and additionally displays, in the graph, the symbol representing the blood pressure related information and the arteriosclerosis related information.

6. The apparatus according to claim 3, wherein the second heartbeat synchronous signal detecting device comprises a heart sound microphone which detects, as the second heartbeat synchronous signal, a heart sound from a heart of the subject as the third portion of the subject.

7. A blood flow amount estimating apparatus, comprising:
a blood pressure related information obtaining device which obtains blood pressure related information that is related to a first blood pressure of a first portion of the subject;
a pulse wave detecting device which detects a pulse wave from the first portion;
an arteriosclerosis related information obtaining device which obtains, based on the pulse wave detected by the pulse wave detecting device, arteriosclerosis related information that is related to arteriosclerosis;
an output device; and
an output-device control device which controls the output device to output a graph which has an axis indicative of blood pressure related information and an axis indicative of arteriosclerosis related information and indicates that blood flow amount changes with respective changes of blood pressure related information and arteriosclerosis related information, and additionally output, in the graph, a symbol representing the blood pressure related information obtained by the blood pressure related information obtaining device and the arteriosclerosis related information obtained by the arteriosclerosis related information obtaining device.

8. The apparatus according to claim 7, wherein the blood pressure related information obtaining device comprises:
a carotid pulse wave detecting device which is adapted to be pressed on a cervical portion of the subject as the first portion of the subject and detects a pressure pulse wave produced from a carotid artery of the cervical portion;
a cuff which is adapted to be worn on a brachial portion of the subject as a second portion of the subject;
a brachial blood pressure determining device which determines a plurality of brachial blood pressure values of the brachial portion, based on a first heartbeat synchronous signal detected from the brachial portion when a pressure in the cuff is changed; and
a cervical blood pressure determining device which determines, as the blood pressure related information, a cervical blood pressure of the cervical portion, based on a minimum magnitude, an area-gravity-center magnitude, and a maximum magnitude of the carotid pulse wave detected by the carotid pulse wave detecting device, and the brachial blood pressure values determined by the brachial blood pressure determining device, and
wherein the pulse wave detecting device comprises the carotid pulse wave detecting device of the blood pressure related information obtaining device.
